(19) United States
(12) Patent Application Publication
(10) Pub. No.: US 2026/0108196 A1
(43) Pub. Date: Apr. 23, 2026

(54) SENSOR GUIDE WIRE

(71) Applicant: Asahi Intecc Co., Ltd., Seto-shi (JP)

(72) Inventor: Hirobumi Watanabe, Irvine, CA (US)

(21) Appl. No.: 19/364,439

(22) Filed: Oct. 21, 2025

Related U.S. Application Data
(60) Provisional application No. 63/709,580, filed on Oct. 21, 2024.

(51) Int. Cl. A61B 5/291; A61B 5/00; A61B 5/06

(52) U.S. Cl. CPC A61B 5/291; A61B 5/061; A61B 5/6851

(57) ABSTRACT

Embodiments of the present disclosure relate to a sensor guidewire and methods for fabricating and using the same. An example sensor guide wire includes a core wire spanning between a first end and a second end, the first end and the second defining a length of the sensor guide wire; a plurality of wires in a helical arrangement around the core wire, wherein: the helical arrangement extending along at least a subset of a length of the core wire; a plurality of electrodes spaced apart from one another along the helical arrangement, wherein: a respective electrode is configured to cover a respective portion of the helical arrangement along the length of the sensor guide wire; the electrode is connected to one of the plurality of wires to enable conduction of signal from the electrode to the wire; and the respective electrode and the wire define an electrophysiological sensor.

### CROSS REFERENCE TO RELATED APPLICATIONS

This application claims the benefit of and priority to U.S. Application No. 63/709,580, filed Oct. 21, 2024, the disclosure of which is incorporated herein by reference in its entirety.

### TECHNICAL FIELD

This application generally relates to sensor guide wires and methods for using the same.

### BACKGROUND

Electrodes may be used to measure bioelectricity within a subject. For example, electrodes may be placed around a subject's skull to measure electrophysiological signals generated by the brain. Greater measurement accuracy and specificity may be obtained by inserting electrodes into the body; however, tortuous structures of the body may increase the difficulty of navigating an electrode to a target site. Further, deployment of multiple electrodes to a target site may be difficult due to limited spatial dimensions at the target site and along an insertion route. Existing approaches have yet to solve the challenge of deploying robust electrophysiological sensors to a desired target site.

### BRIEF SUMMARY

Embodiments of the present disclosure relate to sensor guide wires, kits comprising one or more sensor guide wires, and methods for using the same. An example sensor guide wire of the present disclosure may include a core wire spanning between a first end and a second end, the first end and the second defining a length of the sensor guide wire; a plurality of wires in a helical arrangement around the core wire, wherein: the helical arrangement extending along at least a subset of a length of the core wire; and a plurality of electrodes spaced apart from one another along the helical arrangement, wherein: a respective electrode is configured to cover a respective portion of the helical arrangement along the length of the sensor guide wire; the respective electrode is connected to one of the plurality of wires, the connection enabling conduction of signal from the electrode to the one of the plurality of wires; and the respective electrode and the one of the plurality of wires define an electrophysiological sensor.

In some embodiments, respective electrophysiological sensors defined by respective pairs of the plurality of wires and the plurality of electrodes are electrically independent of one another. In some embodiments, the sensor guide wire further comprises a tip configured to encapsulate the second end of the core wire and respective ends of the plurality of wires proximate to the second to the core wire, wherein: the tip further defines the length of the sensor guide wire. In some embodiments, wherein the helical arrangement is configured to taper from a first diameter to a second diameter toward the tip.

In some embodiments, the core wire is configured to taper from a first diameter to a second diameter toward the tip. In some embodiments, the tip comprises at least one epoxy material. In some embodiments, at least one of the plurality of electrodes comprises a void configured to expose a portion of one of the plurality of wires in the helical arrangement. In some embodiments, the plurality of wires comprises at least 8 wires; and the plurality of electrodes comprises at least 8 electrodes. In some embodiments, the sensor guide wire further comprises the plurality of wires comprises at least 16 wires; and the plurality of electrodes comprises at least 16 electrodes.

In some embodiments, a respective wire of the plurality of wires comprises a platinum-iridium layer configured to impart radiopacity to the helical arrangement. In some embodiments, a thickness of the platinum-iridium layer is at least 25.0 microns. In some embodiments, the respective wire of plurality of wires further comprises a polytetrafluoroethylene (PTFE) layer over the platinum-iridium layer. In some embodiments, a thickness of the PTFE layer is at least 33.0 microns. In some embodiments, a respective wire of the plurality of wires comprises a diameter of at least 0.25 mm. In some embodiments, a respective electrode of the plurality of electrodes comprises a platinum layer; and the platinum layer renders the respective electrode radiopaque.

In some embodiments, the core wire comprises a nickel-titanium alloy. In some embodiments, a cross-section of the core wire comprises a quadrilateral shape. In some embodiments, the plurality of electrodes define respective segments extending along the length of the sensor guide wire; and the sensor guide wire further comprises: at least one additional wire configured to coil around a respective segment of the sensor guide wire. In some embodiments, the length of the sensor guide wire is at least 80.0 mm. In some embodiments, a respective separation distance between the plurality of electrodes is at least 10.0 mm. In some embodiments, a separation distance between a first electrode of the plurality of electrodes and a last electrode of the plurality of electrodes is at least 150.0 mm.

An example sensor guide wire kit may comprise a first sensor guide wire and a second sensor guide wire according to claim, wherein: the plurality of electrodes of the first sensor guide wire are separated from one another by a first separation distance; and the plurality of electrodes of the second sensor guide wire are separated from one another by a second separation distance that is different from the first separation distance; and at least one computing device configured to receive respective signals from the plurality of wires of the first sensor guide wire or the second sensor guide wire. Another example sensor guide wire kit may include at least a first sensor guide wire, a second sensor guide wire, and a third sensor guide wire.

An example method for measuring electrophysiological signals using an embodiment of the sensor guide may comprise navigating the sensor guide wire to through at least one tubular structure of subject to a target site within the subject; positioning at least a subset of the plurality of electrodes proximate to a plurality of sampling sites at the target site, the plurality of sampling sites being spaced apart from one another; measuring respective electrophysiological signals from the plurality of sampling sites via the respective electrode positioned over proximate to the sampling site; and conducting the respective electrophysiological signals from the at least a subset of the plurality of electrodes to a computing device via the one of the plurality of wires that is connected to the electrode.

In some embodiments, the method further comprises monitoring a position of the sensor guide wire within the subject via a radiation-based imaging technique. In some embodiments, the target site comprises a superior sagittal sinus. In some embodiments, the method further comprises positioning the at least a subset of the electrodes comprises: applying torque to the first end of the sensor guide wire to cause a rotation along the length of and at the second end of the sensor guide wire, wherein the plurality of wires are configured to translate the torque along the length of the sensor guide wire.

An example method for fabricating an embodiment of the sensor guide wire may comprise identifying one of the plurality of wires via a thermographic operation; exposing a conductive layer of the identified wire; aligning one of the plurality of electrodes over the core; and forming a conductive connection between the core and the aligned electrode. In some embodiments, the conductive connection comprises at least one of solder, conductive epoxy, or epoxy insulation. In some embodiments, the electrode comprises a void, the electrode being positioned to align the void over the core of the identified wire. In some embodiments, the method further comprises forming the conductive connection through the void of the electrode.

In some embodiments, the thermographic operation comprises: applying a current to the one of the plurality of wires to cause an increase in thermal profile; generating thermal image data in accordance with a portion of the sensor guide wire; and identifying the one of the plurality of wires to which the current is applied based at least in part on the thermal image data. In some embodiments, the method comprises exposing the core of the identified wire via a laser.

### BRIEF DESCRIPTION OF THE FIGURES

Having thus described the embodiments of the disclosure in general terms, reference now will be made to the accompanying drawings, which are not necessarily drawn to scale, and wherein:

FIG. 1 shows a partial perspective view of an example sensor guide wire in accordance with some embodiments of the present disclosure;

FIG. 2 shows a cross section of an example sensor guide wire in accordance with some embodiments of the present disclosure;

FIG. 3 shows a partial perspective view of a helical arrangement in accordance with some embodiments of the present disclosure;

FIG. 4 shows a partial perspective view of an example core wire in accordance with some embodiments of the present disclosure;

FIGS. 5-6 show diagrams of example sensor guide wires in accordance with some embodiments of the present disclosure;

FIG. 7 shows a schematic diagram of cross sections of an example sensor guide wire in accordance with some embodiments of the disclosure;

FIG. 8 shows example techniques for electrode attachment in accordance with some embodiments of the present disclosure;

FIG. 9 shows an example workflow for wire identification and electrode connection in accordance with some embodiments of the present disclosure;

FIG. 10 shows a diagram of an example sensor guide wire deployed to a target site in accordance with some embodiments of the present disclosure;

FIG. 11 illustrates a flowchart of an example fabrication process in accordance with some embodiments of the present disclosure; and

FIG. 12 illustrates a flowchart of an example electrophysiological measurement process in accordance with some embodiments of the present disclosure.

### DETAILED DESCRIPTION

Some embodiments of the present invention will now be described more fully hereinafter with reference to the accompanying drawings, in which some, but not all, embodiments of the invention are shown. Like reference numerals refer to like elements throughout. Indeed, various embodiments of the invention may be embodied in many different forms and should not be construed as limited to the embodiments set forth herein. Rather, these embodiments are provided so that this disclosure will satisfy applicable legal requirements.

As used herein, the term “or” is used in both the alternative and conjunctive sense, unless otherwise indicated. The term “along,” and similarly utilized terms, means near or on, but not necessarily requiring directly on an edge or other referenced location. The terms “approximately,” “generally,” and “substantially” refer to within manufacturing and/or engineering design tolerances for the corresponding materials and/or elements unless otherwise indicated. Thus, use of any such aforementioned terms, or similarly interchangeable terms, should not be taken to limit the spirit and scope of embodiments of the present invention.

As used herein, the term “distal” shall refer to a direction extending toward a target site of a subject (e.g., away from a user of the sensor guide wire). As used herein, the term “proximal” shall refer to a direction extending away from a target site of a subject (e.g., toward a user of the sensor guide wire). For example, a distal end of a sensor guide wire may refer to an end of the sensor guide wire that is directed towards or positioned at a target site within a subject, and a proximal end of the sensor guide may refer to an opposite end of the sensor guide wire that is manipulated by a user (e.g., potentially extending outside of the target site or subject entirely).

The terms “about” or “approximately” as used herein when referring to a measurable value—such as, for example, length, width, height, separation and the like—are meant to encompass variations of ±20%, ±10%, ±5%, ±1%, ±0.5%, or even ±0.1% of the specified amount. A range provided herein for a measurable value may include any other range and/or individual value therein.

### Overview

In general, various embodiments of the present disclosure provide improved designs for sensor guide wires configured to measure electrophysiological signals of a subject. For example, the disclosure provides various embodiments for a sensor guide wire for use in measuring electrophysiological signals of subject's brain via delivery of a plurality of electrodes into the superior sagittal sinus. It will be understood and appreciated that such context is provided by way of example and uses of the sensor guide wire in additional contexts, such as with other medical procedures or target sites, are contemplated and within the scope of the invention. For example, one or more embodiments of the sensor guide wire may be used in other electrophysiology procedures, such as cardiac mapping in which one or more sensor guide wires may be deployed into a subject's heart. The sensor guide wire may be inserted through or into other target sites within the venous system, such as the internal cerebral vein of Galen, cavernous sinus, inferior sagittal sinus, and/or the like. The sensor guide wire may be inserted through or into targets within the arterial system. For example, the sensor guide wire may be deployed through an internal carotid artery to an anterior artery system, medial cerebral artery, arterial branches, and/or the like. As another example, the sensor guide wire may be deployed through a posterior artery system to the basilar artery or portions of the spinal cord system. In some embodiments, the sensor guide wire is deployed into the cerebral subarachnoid system via a void extending through the parietal bone, frontal bone, occipital bone, temporal bone, or other bones of the skull. Additionally, or alternatively, the sensor guide wire may be deployed into the spinal subarachnoid system via a void extending through or between one or more bones of the spinal cord.

In various embodiments, electrophysiology procedures are performed by navigating an electrode through one or more blood vessels to position the electrode within a target site at which desired bioelectrical may be measured. However, the narrow, tortuous structure of blood vessels may present challenges to accurately and safely navigating such devices to a target site. Existing approaches to measuring electrophysiological signals of a subject may demonstrate poor navigability within these and other tortuous structures. For example, an existing system utilizes copper wires coated with insulative materials, which are further incased in a polyimide tube. However, such systems demonstrate undesirable stiffness and limited capacity to transmit torque. As a result, these approaches may be unsuitable for small diameter, tortuous structures such as blood vessels.

Additionally, electrophysiology procedures may benefit from placement of several electrodes into a target site to increase the spatial specificity of bioelectricity measurements. For example, multi-channel electrodes may be utilized for diagnosis of epilepsy in which electrophysical measurements of a plurality of regions of a subject's brain are obtained simultaneously to identify electrical patterns indicative of the condition. While vein-navigable systems based on solid core wires exist, such approaches do not support multi-channel electrophysical measurement. For example, a solid core wire system may include a conductive ribbon embedded in an outer layer of the system; however, the conductive ribbon may limit bioelectrical measurements to a single channel mode, which is insufficient for proper performance of electrophysiological procedures. Thus, existing approaches may fail to provide a multi-channel electrode system with high guide wire performance suitable for deployment in tortuous environments.

To solve these issues and others, example implementations of embodiments of the present application may provide a sensor guide wire with high flexibility and torque transmission and which includes a plurality of electrodes to support multi-channel electrophysiological measurement. In various embodiments, the sensor guide wire includes a multi-wire coil comprising a plurality of individually insulated wires in a helical arrangement. The helical arrangement of wires may demonstrate improved torque transmission performance and flexibility as compared to existing approaches. In this manner, the sensor guide wire may be safely and accurately navigated through tortuous tubular structures, such as bloods vessels.

In various embodiments, the sensor guide wire includes a plurality of electrodes that are each connected to a different wire of the helical arrangement. A respective electrode may surround the helical arrangement and be exposed to the external environment such that electrophysiological signals may be measured via the electrode and conducted to a computing device via the attached wire. In this manner, respective pairs of electrodes and wires of the helical arrangement may embody an independent electrophysiological sensor. The plurality of electrodes may be spaced apart from one another along a length of the helical arrangement such that electrophysical measurements from different regions of a target site may be obtained independently and simultaneously. Additionally, the dual functionality of the wires of the helical arrangement as torque transmitting members and electrical conductors may enable the sensor guide wire to present a reduced diameter that is more optimal for deployment of the apparatus through tubular structures of the body. Further, whereas existing approaches may be limited to measuring an electrophysiological signal from a tip of the device, the present sensor guide wires may enable measurement of multiple electrophysiological signals from different portions extending along the length of the apparatus.

### Example Sensor Guide Wire

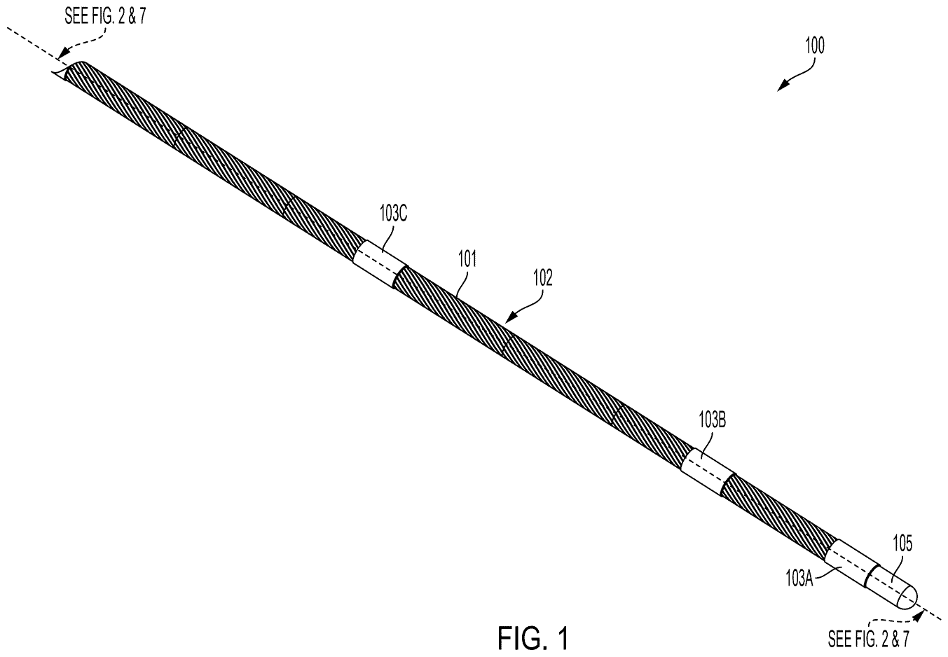

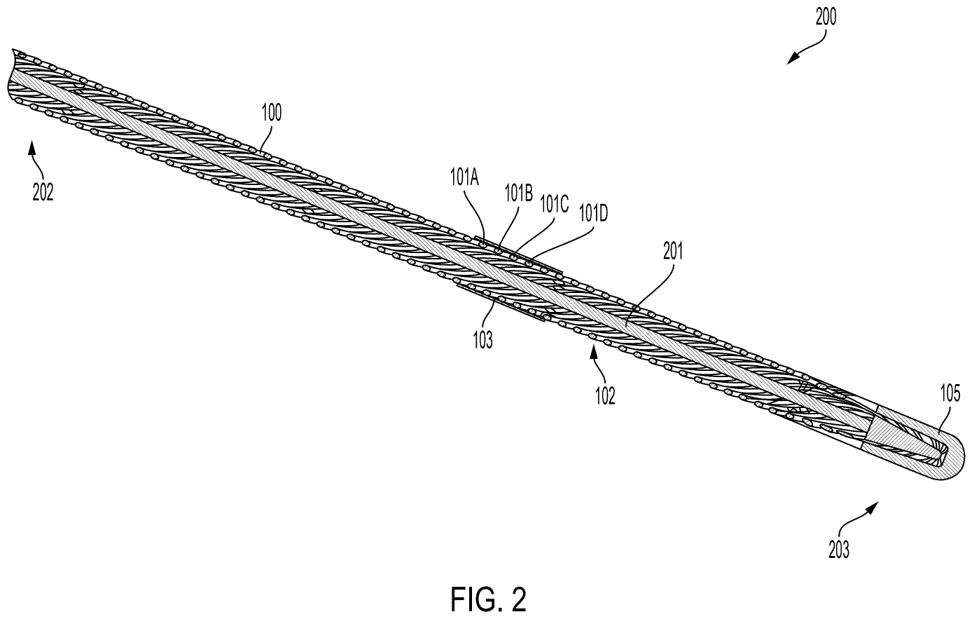

With reference to FIG. 1, a partial perspective view of an example sensor guide wire 100 is illustrated. In some embodiments, the guide wire 100 includes a plurality of wires 101 in a helical arrangement 102. In various embodiments, the helical arrangement 102 provides an optimal balance of torque force, torque response, durability, and rigidity. For example, as compared to existing guide wire approaches, the helical arrangement 102 may demonstrate greater ability to transmit torque along its length (e.g., from a proximal end away from the subject to a distal end toward the subject). As shown in FIG. 2, the guide wire 100 includes a core wire 201 within the helical arrangement 102. For example, the helical arrangement 102 may comprise a central void within which the core wire 201 is located. In some embodiments, the plurality of wires 101 include 8 wires, 16, wires, 24 wires, and/or the like, with greater numbers of wires being contemplated and within the scope of the disclosure.

In various embodiments, respective wires 101 comprise stainless steel material. Additionally, or alternatively, in some embodiments, a wire 101 comprises enamel-coated copper material. In some embodiments, a diameter of the wire 101 is approximately 0.24 to 0.40 mm.

For example, a diameter of the wire 101 may be 0.254 mm. As another example, a diameter of the wire 101 may be 0.355 mm. In some embodiments, a cross-sectional area of the wire 101 is approximately 0.045 to 0.126 square mm. For example, a cross-sectional area of the wire 101 may be 0.05 square mm. As another example, a cross-sectional area of the wire 101 may be 0.099 square mm. In some embodiments, a wire 101 comprises a layer of a platinum-iridium (Pt/Ir) alloy. In various embodiments, the Pt/Ir layer is configured to conduct electrophysiological signals measured by electrodes of the sensor guide wire 100. The Pt/Ir layer may improve the ability of the wire 101 to transmit torque along its length, thereby increasing the overall torque transmission capability of the helical arrangement 102. In some embodiments, a thickness of the Pt/Ir layer is approximately 10.0 to 50.0 microns. For example, a thickness of the Pt/Ir layer may be 25.0 microns. Additionally, the Pt/Ir layer may render the wire 101 radiopaque such that the wire 101 may be visualized via a radiation-based imaging technique, such as X-ray.

In some embodiments, a wire 101 comprises a layer of polytetrafluoroethylene (PTFE). For example, the plurality of wires 101 may be individually coated with a film of PTFE. In some embodiments, the PTFE layer includes a thickness of approximately 20.0 to 60.0 microns. For example, the PTFE layer may include a thickness of 33.0 microns. As another example, the PTFE layer may include a thickness of 4.0 microns. The Pt/Ir layer be covered by the PTFE layer. In this manner, the PTFE layer may electrically insulate the wires 101 from one another, the core wire 201, and the external environment. In some embodiments, the insulation of the wires 101 reduces resistance of the wire as compared to existing approaches, which may result in improved impedance properties. Additionally, the insulation may reduce electrical noise and interference. In some embodiments, the materials of the wire 101 demonstrate respective longitudinal resistivities of approximately 70 microohm-centimeters (stainless steel), 1.7 microohm-centimeters (copper), 10.6 microohm-centimeters (platinum), and 4.71 microohm-centimeters (iridium).

In various embodiments, the sensor guide wire 100 includes a plurality of electrodes 103 spaced apart from one another along the helical arrangement 102. For example, electrodes 103A, 103B, 103C may slide over the helical arrangement 102 and be connected to different wires 101 along the length of the helical arrangement 102. In some embodiments, the sensor guide wire 100 includes approximately 8 to 24 electrodes, with additional numbers of electrodes being contemplated and within the scope of the present disclosure. In some embodiments, the sensor guide wire includes at least 16 electrodes. A respective electrode may be configured to measure and conduct electrophysiological signals from a target site, such as a region of tissue within a subject. In some embodiments, the electrode comprises a tubular shape such that the electrode may slide over and cover a portion of the helical arrangement 102.

In various embodiments, as further shown in the figures and described herein), respective electrodes are conductively connected to different wires 101 such that the electrophysiological signals of an electrode 103 may be conducted along an independent channel. For example, the electrodes 103A, 103B, 103C may be conductively connected to different wires 101. In this manner, embodiments of the sensor guide wire 100 may provide a plurality of channel-independent electrophysiological sensors that are highly navigable within tortuous environments. For example, existing approaches may be limited to single channel measurement (e.g., such existing solutions may be limited to a single electrode contact). The sensor guide wire 100 may demonstrate significant improvements of such approaches by providing 8-channel, 16-channel, or greater electrophysiological measurement capabilities. In doing so, the sensor guide wire 100 may increase the accuracy and specificity of electrophysiological studies, such as epilepsy assessments and electrocardiography investigations. In some embodiments, the electrode includes platinum material that enables the electrode to receive and conduct electrophysiological signals. Additionally, the platinum material may render the electrode radiopaque such that radiation imaging techniques may be used to visualize and track the electrode within a subject.

In some embodiments, the sensor guide wire 100 includes a tip 105 configured to encapsulate respective ends of the plurality of wires 101 and core wire 201. In some embodiments, the tip 105 comprises one or more polymers. For example, the tip 105 may comprise one or more epoxy materials that surround and adhere to distal portions of the wires 101 and core wire 201. The one or more polymers comprising the tip 105 may demonstrate resilience, hardness, and insulative properties. Additionally, or alternatively, in some embodiments, the tip 105 is secured over the plurality of wires 101 and core wire 201 via one or more applied adhesives. In various embodiments, the tip 105 comprises a rounded end 106 configured to reduce likelihood of damaging soft tissues and other structures within a subject.

In various embodiments, the inclusion of platinum in the plurality of wires 101 and plurality of electrodes 103 enable the sensor guide wire 100 to be visualized and tracked throughout navigation within a subject. For example, X-ray imaging may be used to image and inform navigation of the sensor guide wire 100 to a target site.

FIG. 2 shows a cross section 200 of an example sensor guide wire 100. In various embodiments, the core wire 201 extends along a length of sensor guide wire 100. In some embodiments, the core wire 201 and helical arrangement 102 tapers in diameter from a distal portion 202 of the sensor guide wire 100 toward a proximal portion 203 of the sensor guide wire. For example, respective diameters of the core wire 201 and helical arrangement 102 may taper toward the tip 105. In some embodiments, the core wire 201 includes stainless steel material. Additionally, or alternatively, in some embodiments, the core wire 201 includes one or more nickel-titanium alloys. For example, the core wire 201 may include nitinol, and/or the like.

In some embodiments, the electrode 103 is connected to one of the plurality of wires of the helical arrangement 102. For example, the helical arrangement 102 may include wires 101A, 101B, 101C, 101D. In such contexts, four electrodes may be placed over and spaced along the helical arrangement 102. The four electrodes may be connected to the wires 101A, 101B, 101C, 101D, respectively, such that electrophysiological signals received at the electrodes may be conducted along four independent channels (e.g., to a receiving computing device). Thus, the wires 101A-D and four electrodes may define four electrophysiological sensors that are electrically independent of one another. It will be understood and appreciated that the number of wires and electrodes is exemplary and additional wires and electrodes defining additional electrophysiological sensors are contemplated.

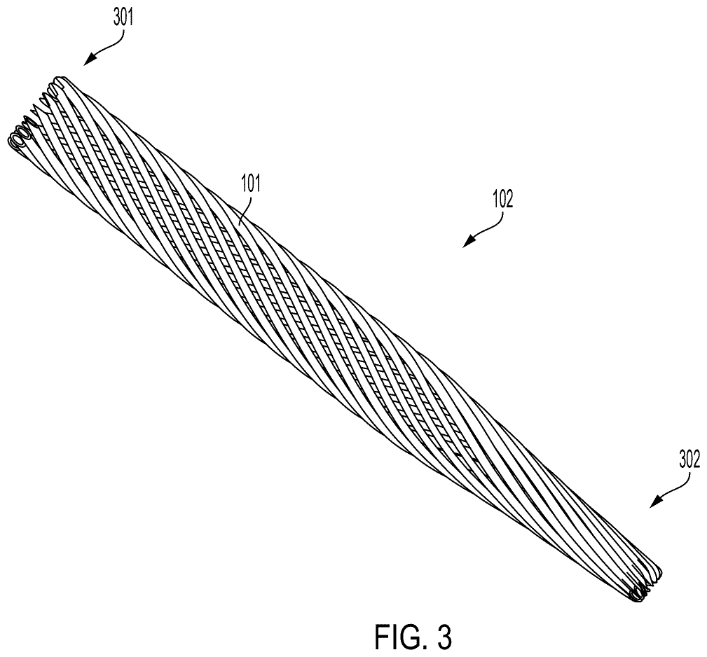

FIG. 3 shows a partial perspective view of a helical arrangement 102. In various embodiments, the helical arrangement 102 tapers from a proximal portion 301 to a distal portion 302. For example, a packing density of the plurality of wires 101 in the helical arrangement 102 may iteratively increase within the distal portion 302. In some embodiments, the plurality of wires 101 terminate at equal lengths at the distal portion 302. Alternatively, in some embodiments, the wires 101 terminate at staggered lengths at the proximal portion 302. For example, the distal end of a first subset of wires 101 may be positioned at a first distance along the length of the sensor guide wire, and the distal end of a second subset of wires 101 may be positioned at a different distance along the length of the sensor guide wire. In such contexts, both the first and second subsets of wires 101 may be encapsulated by a tip 105 as shown in FIGS. 1, 2 and 5-7. In some embodiments, the helical arrangement 102 provides ideal bending stiffness profile to the sensor guide wire, which may improve navigability through tortuous environments including veins, arteries, organs, and/or the like.

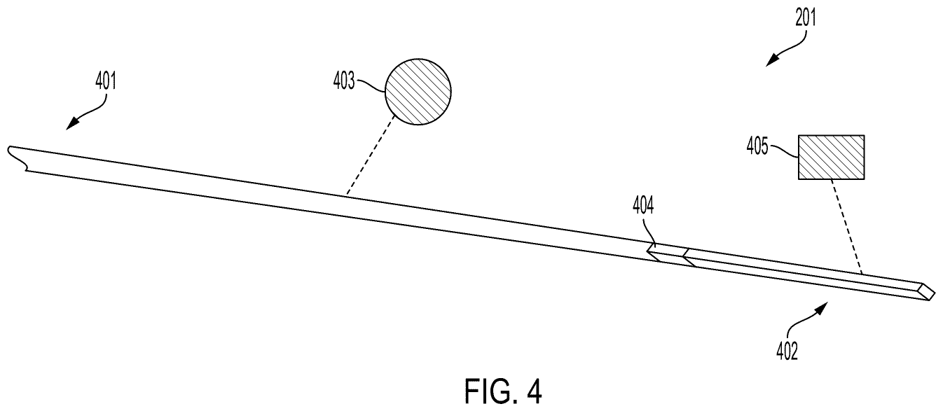

FIG. 4 shows a partial perspective view a core wire 201. In some embodiments, the core wire 201 includes a proximal portion 401 and a distal portion 402. In some embodiments, a cross section 403 of the proximal portion 401 comprises a circular shape. In some embodiments, the core wire 201 includes a transition 404 between the proximal portion 401 and the distal portion 402. In various embodiments, at the transition 404, the shape of the cross section of the core wire 201 shifts from a circular shape to a quadrilateral shape. For example, a cross section 405 of the distal portion 402 may include a quadrilateral shape. Alternatively, the cross section 405 may include other angular polygon shapes (e.g., hexagon, heptagon, triangle, octagon, and/or the like).

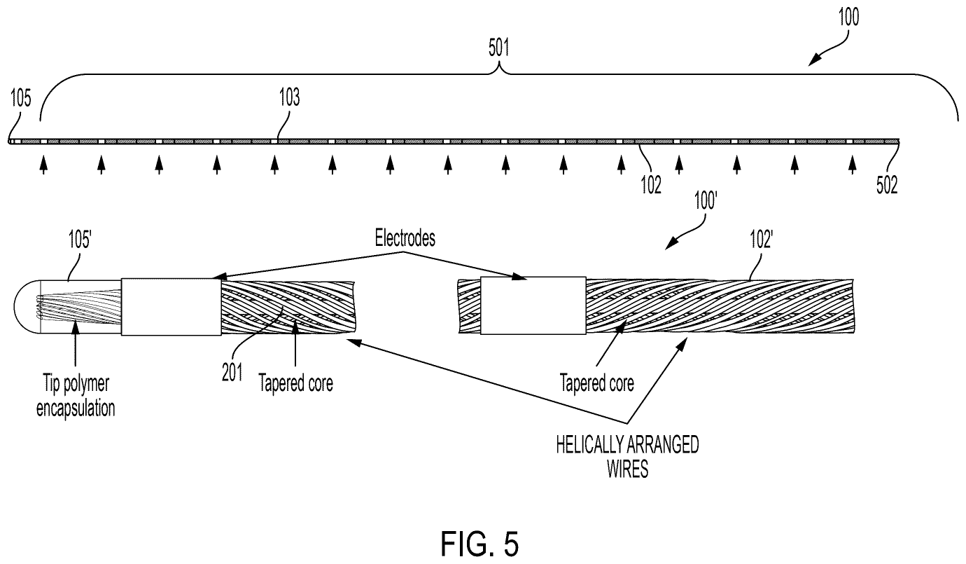

FIG. 5 shows a diagram of an example sensor guide wire 100, 100′. As shown, an example embodiment of the sensor guide wire 100 includes 16 electrodes 103 extending along a length 501 of a portion of the helical arrangement 102 (e.g., the portion being proximate to the tip 105). In some embodiments, the portion of the helical arrangement 102 that is nearest to the tip 105 is referred to as a “distal portion” of the helical arrangement 102. In such contexts, a portion of the sensor guide wire 100 extending from the tip 105 to an end 502 of the distal portion of the helical arrangement 102 may be referred to as a “distal portion” of the sensor guide wire 100. In some embodiments, the core wire of the sensor guide wire 100 extends past the distal portion of the helical arrangement 102.

In some embodiments, the length 501 of the distal portion of the helical arrangement 102 is approximately 60.0 to 160.0 mm. For example, the 16 electrodes 103 of the sensor guide wire 100 shown in FIG. 5 may span 80.0 mm, and a respective separation distance between the plurality of electrodes 103 may be 5.0 mm. In another example, the sensor guide wire 100 may include 8 electrodes 103 spaced apart by a separation of 10.0 mm such that the plurality of electrodes 103 span 80.0 mm. In another example, the sensor guide wire 100 may include 16 electrodes 103 spaced apart by a separation of 10.0 mm such that the plurality of electrodes span 160.0 mm (e.g., a separation distance between a most-proximal electrode and a most-distal electrode being 150.0 mm).

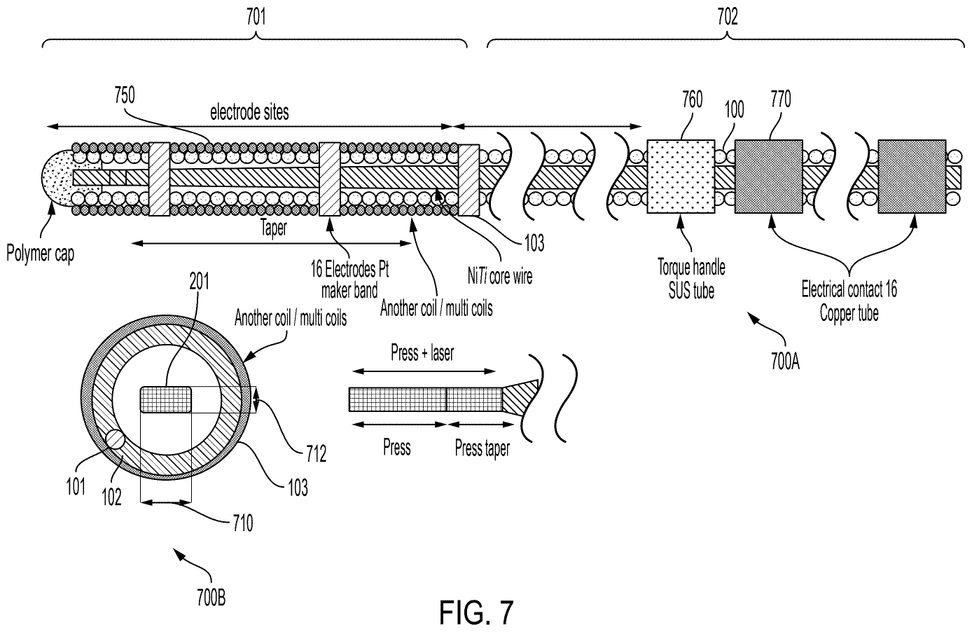

In various embodiments, the tip 105, 105′ encapsulates distal ends of the core wire 201 and plurality of wires of the helical arrangement 102, 102′, In some embodiments, the helical arrangement 102′ tapers in diameter toward the tip 105′. For example, over the length 501, the helical arrangement 102′ may taper from a first diameter of 0.218 to a second diameter of 0.0864 mm. In some embodiments, the core wire 201 tapers in one or more dimensions toward the tip 105′ and over at least the length 501 of the distal portion of the helical arrangement 102. For example, a cross section of the core wire 201 may comprise a quadrilateral shape defining a width and height. In such contexts, the core wire 201 may taper in width and/or height toward the tip 105′ and over the length 501 (e.g., 80.0 mm), or a further along the length of the sensor guide wire 100, 100′, such as 100.0 mm. In some embodiments, at the tip 105′, the core wire 201 includes a terminal width 710 of approximately 0.05 mm to 0.080 mm and a terminal height 712 of approximately 0.03 mm to 0.07 mm. For example, the terminal width 710 may be 0.068 mm and the terminal height 712 may be 0.044 mm (see FIG. 7). In some embodiments, the taper of the core wire 201 is 0.180 mm of width and/or height per mm of length.

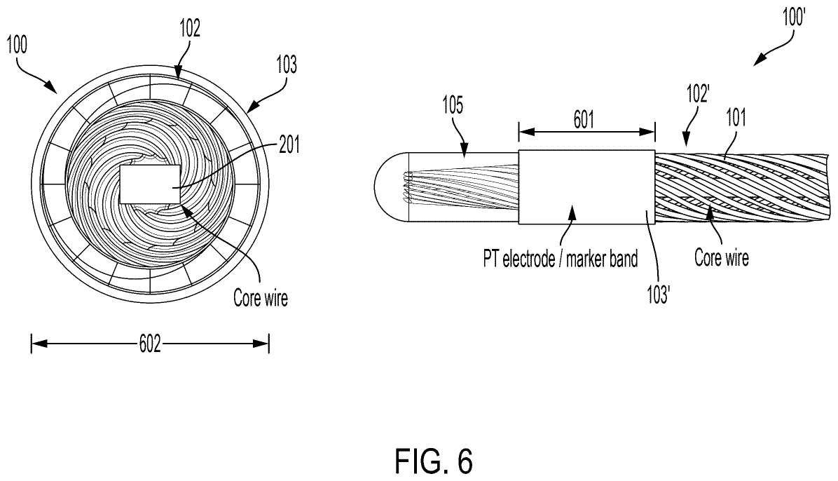

FIG. 6 shows another diagram of the example sensor guide wire 100, 100′. In some embodiments, a length 601 (FIG. 6) of the electrode 103 is approximately 0.5 to 4.0 mm. For example, the length 601 may be 0.5 mm. In another example, the length 601 may be 2.0 mm, 3.0 mm, and/or the like. In this manner, the electrode 103 may present sufficient outer surface area (e.g., approximately 0.4 to 3.4 square millimeters, and/or the like) for interfacing with bodily fluid such that measurements of impedance may be obtained. Further, the diminutive length of the electrode 103 may increase the flexibility of the sensor guide wire, thereby improving navigability through tortuous environments. In some embodiments, the sensor guide wire 100 includes a plurality of electrodes 103 of different lengths 601. In some embodiments, an outer diameter 602 of the electrode 103 is approximately 0.2 to 0.5 mm. For example, the outer diameter 602 of the electrode 103 may be 0.27 mm. In some embodiments, a thickness of the electrode 103 is approximately 10.0 to 50.0 microns. The thickness may comprise a distance between an outer diameter and an inner diameter of the electrode 103. For example, a thickness of the electrode 103 may be 10.0 microns. In some embodiments, the electrode 103 demonstrates an impedance of approximately 80 to 120 kiloohms. For example, the electrode 103 may comprise an impedance of 100 kiloohms.

FIG. 7 shows a schematic diagram of cross sections 700A, 700B of an example sensor guide wire 100. The cross section 700A may be taken along a longitudinal axis of the sensor guide wire 100. The cross section 700B may be taken along a transversal axis orthogonal to the longitudinal axis. In some embodiments, the sensor guide wire 100 includes a distal portion 701 and a proximal portion 702. In some embodiments, a length of the distal portion 701 is at least 80.0 mm, with greater or lesser lengths being contemplated and within the scope of the present disclosure. In some embodiments, a length of the proximal portion 702 is at least 520.0 mm, with greater or lesser lengths being contemplated and within the scope of the present disclosure.

The distal portion 701 may comprise the plurality of electrodes 103. For example, the distal portion 701 may include 16 electrodes spaced apart from one another along the helical arrangement 102. In some embodiments, along the proximal portion 702, the core wire 201 includes a diameter of approximately 0.1 to 0.3 mm. For example, along the proximal portion 702, the core wire may include a diameter of 0.17 mm. As shown in FIG. 6, the cross section of the core wire 201 may transition from a circular shape in the proximal portion 702 to a quadrilateral shape in the distal portion 701. In some embodiments, along the distal portion 701, the dimensions of the core wire 201 taper toward a terminal width 710 and terminal height 712. In one example, the terminal width 710 and terminal height 712 measure 0.068 mm and 0.044 mm, respectively. In some embodiments, the core wire 201 is laser annealed to remove residual stresses, which may be generating during pressing processes that are performed to taper the core wire 201.

In some embodiments, a respective wire 101 includes a cross section including an ovoid or circular shape. In some embodiments, a diameter of the wire 101, including a Pt/Ir layer, is approximately 0.15 to 0.40 mm. For example, a diameter of the wire 101, including a Pt/Ir layer is 0.25 mm. In some embodiments, the diameter of the wire 101, further including an outer PTFE layer, is approximately 0.2 to 0.5 mm. For example, the diameter of the wire 101, further including an outer PTFE layer, may be 0.33 mm. In some embodiments, at distal end of the distal portion 701 of the sensor guide wire 100, the helical arrangement 102 includes an outer diameter of approximately 0.2 to 0.4 mm and an inner diameter of approximately 0.1 to 0.3 mm. For example, at distal end of the distal portion 701 of the sensor guide wire 100, the helical arrangement 102 may include an outer diameter of 0.250 mm and an inner diameter of 0.186 mm.

In some embodiments, the distal portion 701 includes one or more additional wires 750 configured to coil around respective segments of the helical arrangement 102. In some embodiments, the wire 750 increases torque transmission along the sensor guide wire 100. In some embodiments, the wire 750 is configured to standardize an overall outer diameter of the sensor guide wire 100 to a constant value. For example, the distal portion 701 may include one or more wires 750 to cause the distal portion 701 to demonstrate an overall outer diameter that is approximately equal to the overall outer diameter of the proximal portion of the sensor guide wire 100. In some embodiments, the distal portion 701 includes a plurality of wires 750 coiled around respective segments of the helical arrangement 102 between the electrodes 103 that are positioned along the helical arrangement 102. In some embodiments, the wire 750 includes nickel-titanium alloys, stainless steel, and/or the like. In some embodiments, the wire 750 is electrically insulated from the electrodes 103 and wires 101 of the helical arrangement 102. For example, the wire 750 may be incased within polyimide tubing (e.g., the wire 750 being adhered within via one or more epoxies).

In some embodiments, the proximal portion 702 of the sensor guide wire 100 includes one or more torque handles 760 by which a user may apply torque to the sensor guide wire 100. In such contexts, the helical arrangement 102, core wire 201, and/or the like may transfer the torque along the length of the sensor guide wire 100 from the proximal portion 702 to the end of the distal portion 701. In doing so, the sensor guide wire 100 may be navigated through tortuous environments within a subject. In some embodiments, the torque handle 760 is secured over a segment of the helical arrangement 102. For example, the torque handle 760 may be adhered to the outer PTFE layer of the wires 101 via epoxy. Additionally, or alternatively, in some embodiments, the torque handle 760 is adhered to the core wire 201 via epoxy. In various embodiments, the torque handle 760 is configured to withstand gripping pressures of a user to enable the user to apply torque to the sensor guide wire 100. In some embodiments, the torque handle 760 comprises stainless steel. In some embodiments, the torque handle 760 may include an outer diameter of approximately 0.2 to 0.5 mm. For example, the torque handle 760 may include an outer diameter of approximately 0.35 mm.

In some embodiments, the sensor guide wire 100 includes a plurality of electrical contacts 770. A respective electrical contact 770 may be conductively coupled to one of the plurality of wires 101. In this manner, the electrical contact 770 may further conduct electrophysiological signals conducted by the wire 101 to a computing device for analysis. For example, the sensor guide wire 100 may include 16 wires 101 and 16 electrical contacts 770 that are each conductively coupled to one of the 16 wires. In doing so, the sensor guide wire 100 may provide enable electrophysiological measurement over 16 independent channels. In some embodiments, an electrical contact 770 comprises a tubular shape such that the electrical contact 770 may slide over the helical arrangement 102 and be positioned over a segment thereof. In some embodiments, an electrical contact 770 includes a diameter of approximately 0.2 to 0.5 mm. For example, the electrical contact 770 may include a diameter of 0.35 mm. In various embodiments, the techniques, processes, and workflows described herein for conductively connecting an electrode 103 to a wire 101 may also be applied to conductively connect an electrical contact 770 to the wire 101. In some embodiments, the electrical contact 770 includes one or more copper-comprising materials.

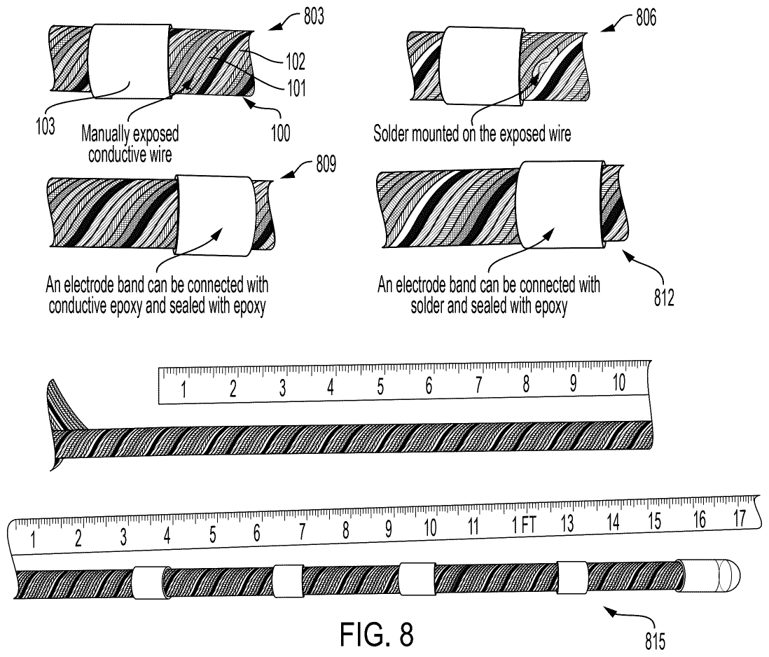

FIG. 8 shows example techniques for attaching an electrode 103 to a wire 101 of the sensor guide wire 100. In some embodiments, the electrode 103 is slid over the helical arrangement 102. In some embodiments, a Pt/IR layer of the wire 101 is exposed via removal of a portion of the outer PTFE layer. For example, the PTFE layer may be manually exposed (indicium 803), laser-removed, machine-cut, and/or the like. In some embodiments, solder material is deposited to the exposed Pt/Ir layer (indicium 806). For example, gold-based solder may be deposited onto the Pt/Ir layer. Alternatively, in some embodiments, conductive epoxy is deposited onto the exposed Pt/Ir layer in place of solder material (indicium 809). In some embodiments, conductive epoxy is applied over the deposited solder material (indicium 812). The electrode 103 may be slid over the deposited solder material (or conductive epoxy) such that the conductive epoxy adheres to and secures the position of the electrode 103 (indicium 812). In this manner, the conductive epoxy and solder may conductively connect the electrode 103 to the wire 101 such that electrophysiological signals measured by the electrode 103 may be conducted along the wire 101 independent of other electrophysiological signals measured by other electrodes 103. In some embodiments, the electrode 103 may be sealed with additional conductive or non-conductive epoxy to prevent shifting or disconnection of the electrode 103. In various embodiments, the technique may be repeated to couple a plurality of electrodes 103 to different wires 101 of the helical arrangement 102 (indicium 815).

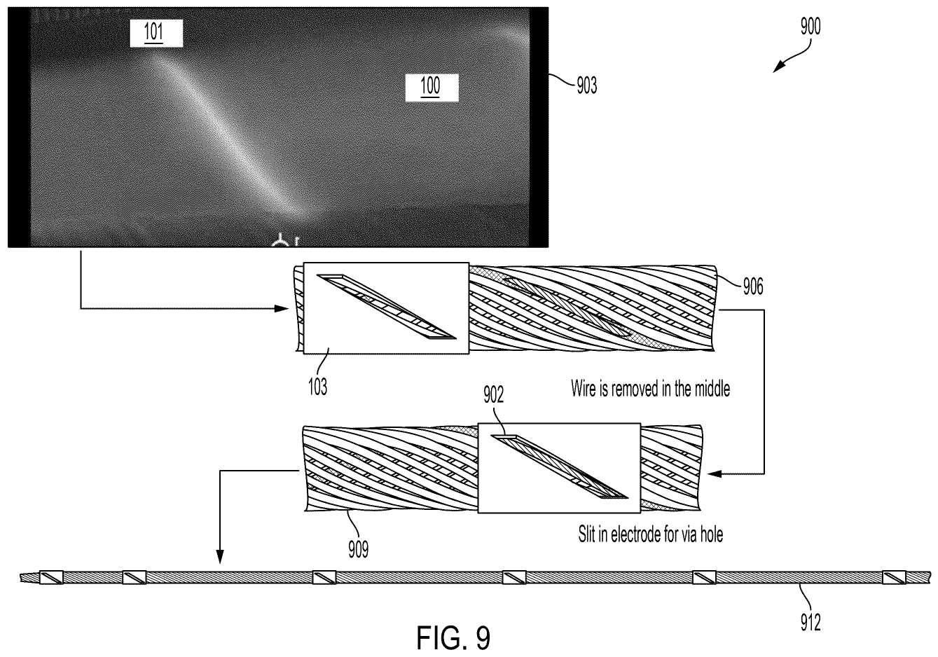

FIG. 9 shows an example workflow for wire identification and electrode connection. In some embodiments, a quantity of wires 101 in the sensor guide wire 100 increases the challenge of identifying a target wire for connection to an electrode 103. In various embodiments, a thermographic operation is performed to increase a thermal profile of the target wire 101 such that the wire is identifiable and differentiable from other wires in thermal data-based visualizations of the sensor guide wire 100. In some embodiments, at operation 903, a current is applied to the target wire 101, which may result in a rise in temperature of the target wire 101. In some embodiments, operation 903 further includes thermally imaging the sensor guide wire 100. The rise in temperature of the target wire 101 may increase its thermal profile such that the wire is identifiable as a peak in the thermal data. For example, by passing a current through a target wire 101 or subset of wires 101, the rise in temperature and thermal profile may be limited to the targets. In this manner, the target wire may be identified with precision and accuracy.

In some embodiments, at operation 906, the workflow 900 includes removing an outer PTFE layer of the identified wire 101 via a laser, cutting mechanism, and/or the like. In this manner, the conductive Pt/Ir layer of the wire 101 may be exposed. In some embodiments, the electrode 103 includes a void 902 that extends through an outer and inner surface of the electrode. In some embodiments, the void 902 is formed via laser. In various embodiments, at operation 909, the workflow 900 includes sliding the electrode 103 over the target wire 101 such that the void 902 is aligned with the exposed Pt/Ir layer. In some embodiments, the workflow 900 includes applying solder and/or conductive epoxy through the void 902 such that the electrode 103 is secured over and conductively connected to the target wire 101. In some embodiments, the workflow 900 includes sealing the void 902 epoxy, gold-based solder, and/or the like. In some embodiments, the workflow 900 further includes polishing the electrode 103 to achieve surface luster (also referred to as “flash”). In various embodiments, the workflow 900 includes repeating operations 903-909 to fabricate a plurality of independent electrophysiological sensors comprised of pairs of electrodes 103 and wires 101. In some embodiments, the workflow 900 is further performed to conductively connect electronic contacts to the wires 101 at a proximal portion of the sensor guide wire 100. Further, one or more computing devices may be connected to the electronic contacts to enable collection, storage, and/or analysis of the conducted electrophysiological signals.

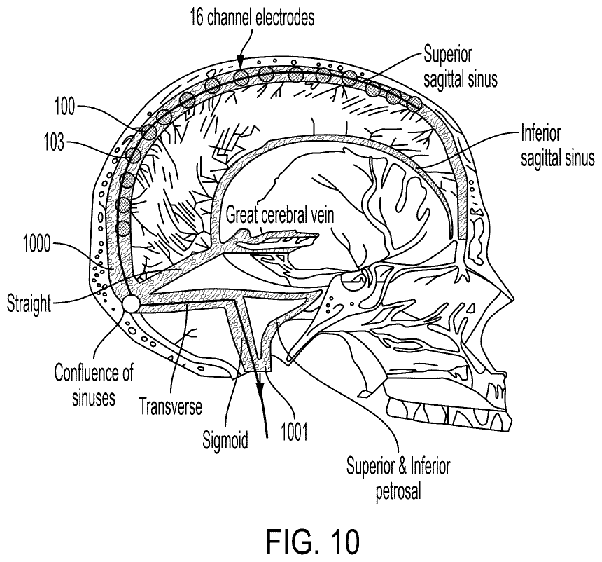

FIG. 10 shows an example sensor guide wire 100 deployed to a target site 1000. It will be understood and appreciated that the illustration shown in FIG. 10 is exemplary in nature and is not intended to represent or limit proportions, angular arrangements, and/or the like of the sensor guide wires described herein. In some embodiments, the sensor guide wire 100 may be navigated through a plurality of tubular structures of a circulatory system 1001 of a subject to deploy a distal portion of the sensor guide wire 100 to a target site 1000 by which electrophysiological signals of the subject's brain may be measured. In some embodiments, the target site 1000 comprises a superior sagittal sinus of the subject. In some embodiments, the plurality of electrodes 103 of the sensor guide wire 100 enable multi-channel electrophysiological measurement of brain activity. For example, the sensor guide wire 100 may include 16 electrically independent and spatially separated electrodes 103 such that electrophysiological signals from a plurality of sampling sites within the brain may be measured and conducted to a computing device simultaneously. In doing so, the sensor guide wire 100 may demonstrate technical advantages over existing approaches to electrophysiological measurement by increasing the number and spatial specificity of the measurements.

### Example Methods of Fabrication and Use

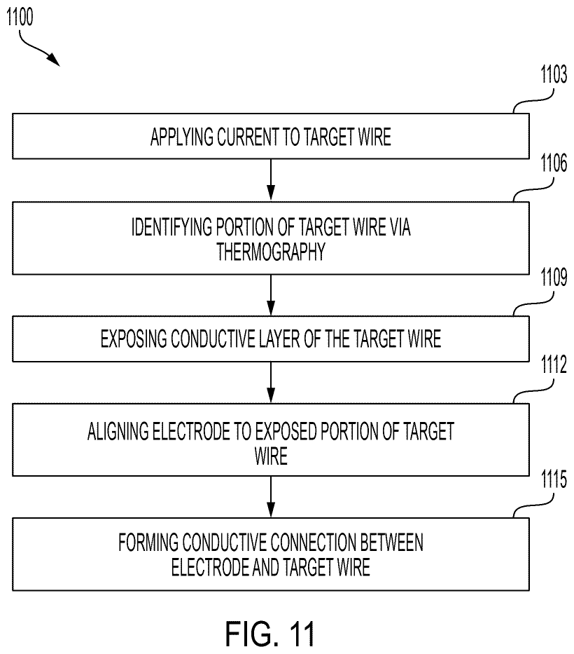

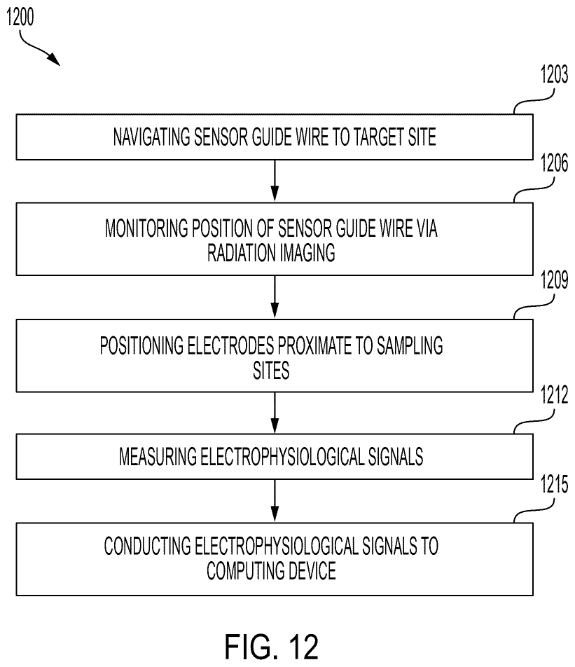

Having described example sensor guide wires in accordance with the disclosure, example processes of the disclosure will now be discussed. It will be appreciated that the flowcharts depict example processes for fabricating and using sensor guide wires described herein. For example, a fabrication process 1100 depicted in the flowchart of FIG. 11 and described herein may be performed using one or more sensor guide wires 100, as shown in FIGS. 1, 2, and 5-9, respectively, and described herein. As another example, an electrophysiological measurement process 1200 depicted in the flowchart of FIG. 12 and described herein may be performed using one or more sensor guide wires 100. In some embodiments, one or more processes are performed using a kit comprising one or more sensor guide wires. For example, the process 1200 may be performed using a kit comprising one or more sensor guide wires 100 and/or the like. As another example, in a context of mapping electrophysiological activity of a brain, the process 1200 may be performed in multiple iterations using a kit comprising a first sensor guide wire 100, a second guide wire 100, and a third guide wire 100. In such contexts, the first sensor guide wire 100 may be deployed into the superior sagittal sinus or inferior sagittal sinus, the second sensor guide wire 100 may be deployed to the left transverse sinus, and the third sensor guide wire may be deployed into the right transverse sinus. In some embodiments, the kit further includes one or more computing devices configured to receive signals from the plurality of wires of a sensor guide wire.

In some embodiments, the kit comprises at least a first sensor guide wire and a second sensor guide wire. The first sensor guide wire may comprise a plurality of electrodes spaced apart from one another by a first separation distance. The second sensor guide wire may comprise a plurality of electrodes spaced apart from one another by a second separation distance The second separation distance may be less than, equal to, or greater than the first separation distance. Additionally, or alternatively, in some embodiments, a first sensor guide wire of the kit includes a first quantity of electrodes, and a second sensor guide wire of the kit includes a second quantity of electrodes that is greater than or less than the first quantity of electrodes. In various embodiments, a kit includes multiple sensor guide wires of varying diameter.

The depicted blocks indicate operations of each process. Such operations may be performed in any of a number of ways, including, without limitation, in the order and manner as depicted and described herein. In some embodiments, one or more blocks of any of the processes described herein occur in-between one or more blocks of another process, before one or more blocks of another process, in parallel with one or more blocks of another process, and/or as a sub-process of a second process. Additionally, or alternatively, any of the processes in various embodiments include some or all operational steps described and/or depicted, including one or more optional blocks in some embodiments. With regard to the flowcharts illustrated herein, one or more of the depicted block(s) in some embodiments is/are optional in some, or all, embodiments of the disclosure. It should be appreciated that one or more of the operations of each flowchart may be combinable, replaceable, and/or otherwise altered as described herein.

FIG. 11 illustrates a flowchart depicting operations of an example process 1100 for fabricating a sensor guide wire 100. For example, the process 2400 may be performed to conductively couple a plurality of electrodes 103 to a plurality of wires 101 of a helical arrangement 102. In doing so, a plurality of electrically independent electrophysiological sensors may be defined by respective pairs of electrodes 103 and wires 101.

In some embodiments, at block 1103, the process 1100 includes applying a current to a target wire 101. In doing so, the temperature of the target wire 101 may be raised without substantial rise of temperature in other wires due to the wires being insulated from one another. In some embodiments, at block 1106, the process 1100 includes identifying a portion of the target wire 101 via thermography. For example, a thermographic operation may be performed to generate thermal image data of the sensor guide wire 100. The target wire 101, and portion thereof that will be connected to an electrode, may be identified based on the thermal image data, in which the thermal profile of the target wire may be distinguishable as a peak or outlier.

In some embodiments, at block 1109, the process 1100 includes exposing a conductive layer of the target wire along a portion thereof. For example, a section of outer insulative material (e.g., PTFE, and/or the like) may be removed via laser or mechanical means. In this manner, a sublayer of conductive material (e.g., Pt/Ir coating) may be exposed. In some embodiments, at block 1112, the process 1100 includes aligning an electrode 103 over the exposed portion of the target wire 101. For example, an electrode 103 may be arranged over the helical arrangement 102 such that a void of the electrode 103 is aligned over the exposed portion of the target wire 101. In some embodiments, at block 1115, the process 1100 includes forming a conductive connection between the electrode 103 and the exposed portion of the target wire 101. For example, solder, conductive epoxy, and/or the like may be applied via the void of the electrode 103. The solder, conductive epoxy, and/or the like may conductively connect and secure the electrode 103 to the target wire 101. In some embodiments, the process 1100 includes polishing the void of the electrode 103 to achieve a flush surface free of burrs, pits, and/or the like. In various embodiments, the process 1100 may be repeated to form independent conductive connections between additional electrodes 103 and wires 101. Further, in some embodiments, the process 1100 may be repeated at a proximal end of the sensor guide wire 100 to form conductive connections between the plurality of wires and a plurality of electrical contacts, respectively.

FIG. 12 illustrates a flowchart depicting operations of an example process 1200 for measuring electrophysiological signals from a target site. For example, the process 1200 may be performed to measure a plurality of electrophysiological signals from different regions of a brain. As another example, the process 1200 may be performed to generate a mapping of electrical phenomena throughout a heart.

In some embodiments, the process 1200 includes conductively coupling a computing device to a plurality of electrical contacts at a proximal portion of the sensor guide wire 100. For example, a plurality of independent electrical connections may be formed between a computing device and the electrical contacts. In this manner, the computing device may receive and record electrophysiological signals over a plurality of channels, which may correspond to different sampling sites of a target site.

In some embodiments, at block 1203, the process 1200 includes navigating the sensor guide wire 100 to a target site. For example, leading with a distal portion, the sensor guide wire 100 may be inserted into and navigated through one or more blood vessels of a subject to deploy the distal portion of the sensor guide wire 100 to a target site, such as a superior sagittal sinus or one or more chambers, valves, and/or the like of the heart. In some embodiments, block 1203 is performed in parallel with the operations of block 1206. In some embodiments, the sensor guide wire 100 is inserted into an artery of a subject via an incision and navigated through the artery to a particular section thereof or to a secondary blood vessel (e.g., other artery, vein, and/or the like).

In some embodiments, at block 1206, the process 1200 includes monitoring a position of the sensor guide wire 100 within the subject via one or more radiation-based imaging techniques. For example, the radiopaque elements of the sensor guide wire 100 (e.g., wires of the helical arrangement 102, electrodes 103, and/or the like) may be imaged in real-time via X-ray, fluoroscopy, and/or the like. In some embodiments, a user applies torque at the proximal portion of the sensor guide wire 100 based at least in part on the monitored position. The torque may be transmitted from the proximal portion to the distal portion of the sensor guide wire 100 to navigate the apparatus through tortuous environments within the subject.

In some embodiments, at block 1209, the process 1200 includes positioning respective electrodes 103 of the sensor guide wire 100 proximate to a plurality of sampling sites at the target site. For example, via application and transmission of torque, the orientation of the sensor guide wire 100 may be adjusted to position electrodes 103 in line with respective sampling sites, such as different regions of the superior sagittal sinus, heart, and/or the like. In some embodiments, at block 1212, the process 1200 includes measuring respective electrophysiological signals at the sampling sites via the plurality of electrodes 103. In some embodiments, at block 1215, the process 1200 includes conducting the electrophysiological signals to a computing device via the independent conductive connections between the electrodes 103 and respective wires 101 of the helical arrangement 102. The process 1200 may further include reading the respective electrophysiological signals from the plurality of wires 101 via a plurality of electrical contacts in the proximal portion of the sensor guide wire 100. In this manner, the sensor guide wire 100 may be utilized to obtain a plurality of independent, spatially varied measurements of bioelectrical phenomena within tortuous environments of the subject.

### CONCLUSION

While some embodiments described herein relate to sensor guide wires for multi-channel measurement of electrophysiological signals within tortuous environments, one of ordinary skill in the art will appreciate that the teachings herein may also apply to a wide range of medical procedures and apparatuses. The embodiments described herein may also be scalable to accommodate at least the aforementioned applications. Various components of embodiments described herein can be added, removed, reorganized, modified, duplicated, or the like as one skilled in the art would find convenient and/or necessary to implement a particular application in conjunction with the teachings of the present disclosure. In some embodiments, specialized features, characteristics, materials, components, and/or equipment may be applied in conjunction with the teachings of the present disclosure as one skilled in the art would find convenient and/or necessary to implement a particular application.

Moreover, many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of any appended claims. Moreover, although the foregoing descriptions and the associated drawings describe example embodiments in the context of certain example combinations of elements and/or functions, it should be appreciated that different combinations of elements and/or functions can be provided by alternative embodiments without departing from the scope of any appended claims. In this regard, for example, different combinations of elements and/or functions than those explicitly described above are also contemplated as can be set forth in some of any appended claims. Although specific terms are employed herein, they are used in a generic and descriptive sense only and not for purposes of limitation.

That which is claimed is:

1. A sensor guide wire, comprising:
a core wire spanning between a first end and a second end, the first end and the second defining a length of the sensor guide wire;
a plurality of wires in a helical arrangement around the core wire, wherein:
the helical arrangement extending along at least a subset of a length of the core wire; and
a plurality of electrodes spaced apart from one another along the helical arrangement, wherein:
a respective electrode is configured to cover a respective portion of the helical arrangement along the length of the sensor guide wire;
the respective electrode is connected to one of the plurality of wires, the connection enabling conduction of signal from the electrode to the one of the plurality of wires; and
the respective electrode and the one of the plurality of wires define an electrophysiological sensor.

2. The sensor guide wire of claim 1, wherein:
respective electrophysiological sensors defined by respective pairs of the plurality of wires and the plurality of electrodes are electrically independent of one another.

3. The sensor guide wire of claim 1, further comprising:
a tip configured to encapsulate the second end of the core wire and respective ends of the plurality of wires proximate to the second to the core wire, wherein:
the tip further defines the length of the sensor guide wire.

4. The sensor guide wire of claim 3, wherein:
wherein the helical arrangement is configured to taper from a first diameter to a second diameter toward the tip.

5. The sensor guide wire of claim 4, wherein:
the core wire is configured to taper from a first diameter to a second diameter toward the tip.

6. The sensor guide wire of claim 3, wherein:
the tip comprises at least one epoxy material.

7. The sensor guide wire of claim 1, wherein:
at least one of the plurality of electrodes comprises a void configured to expose a portion of one of the plurality of wires in the helical arrangement.

8. The sensor guide wire of claim 1, wherein:
the plurality of wires comprises at least 8 wires; and
the plurality of electrodes comprises at least 8 electrodes.

9. The sensor guide wire of claim 1, wherein:
the plurality of wires comprises at least 16 wires; and
the plurality of electrodes comprises at least 16 electrodes.

10. The sensor guide wire of claim 1, wherein:
a respective wire of the plurality of wires comprises a platinum-iridium layer configured to impart radiopacity to the helical arrangement.

11. The sensor guide wire of claim 10, wherein:
a thickness of the platinum-iridium layer is at least 25.0 microns.

12. The sensor guide wire of claim 10, wherein:
the respective wire of plurality of wires further comprises a polytetrafluoroethylene (PTFE) layer over the platinum-iridium layer.

13. The sensor guide wire of claim 12, wherein:
a thickness of the PTFE layer is at least 33.0 microns.

14. The sensor guide wire of claim 1, wherein:
a respective wire of the plurality of wires comprises a diameter of at least 0.25 mm.

15. The sensor guide wire of claim 1, wherein:
a respective electrode of the plurality of electrodes comprises a platinum layer; and
the platinum layer renders the respective electrode radiopaque.

16. The sensor guide wire of claim 1, wherein:
the core wire comprises a nickel-titanium alloy.

17. The sensor guide wire of claim 1, wherein:
a cross-section of the core wire comprises a quadrilateral shape.

18. The sensor guide wire of claim 1, wherein:
the plurality of electrodes define respective segments extending along the length of the sensor guide wire; and
the sensor guide wire further comprises:
at least one additional wire configured to coil around a respective segment of the sensor guide wire.

19. The sensor guide wire of claim 1, wherein:
the length of the sensor guide wire is at least 80.0 mm.

20. The sensor guide wire of claim 1, wherein:
a respective separation distance between the plurality of electrodes is at least 10.0 mm.

21. The sensor guide wire of claim 1, wherein:
a separation distance between a first electrode of the plurality of electrodes and a last electrode of the plurality of electrodes is at least 150.0 mm.

22. A method for measuring electrophysiological signals using the sensor guide wire of claim 1, the method comprising:
navigating the sensor guide wire to through at least one tubular structure of subject to a target site within the subject;
positioning at least a subset of the plurality of electrodes proximate to a plurality of sampling sites at the target site, the plurality of sampling sites being spaced apart from one another;
measuring respective electrophysiological signals from the plurality of sampling sites via the respective electrode positioned over proximate to the sampling site; and
conducting the respective electrophysiological signals from the at least a subset of the plurality of electrodes to a computing device via the one of the plurality of wires that is connected to the electrode.

23. The method of claim 22, further comprising:
monitoring a position of the sensor guide wire within the subject via a radiation-based imaging technique.

24. The method of claim 22, wherein:
the target site comprises a superior sagittal sinus.

25. The method of claim 22, wherein:
positioning the at least a subset of the electrodes comprises:
applying torque to the first end of the sensor guide wire to cause a rotation along the length of and at the second end of the sensor guide wire, wherein the plurality of wires are configured to translate the torque along the length of the sensor guide wire.

26. A method for fabricating the sensor guide wire of claim 1, comprising:
identifying one of the plurality of wires via a thermographic operation;
exposing a conductive layer of the identified wire;
aligning one of the plurality of electrodes over the core; and
forming a conductive connection between the core and the aligned electrode.

27. The method of claim 26, wherein:
the conductive connection comprises at least one of solder, conductive epoxy, or epoxy insulation.

28. The method of claim 26, wherein:
the electrode comprises a void, the electrode being positioned to align the void over the core of the identified wire; and
the method further comprising:
forming the conductive connection through the void of the electrode.

29. The method of claim 26, wherein:
the thermographic operation comprises:
applying a current to the one of the plurality of wires to cause an increase in thermal profile;
generating thermal image data in accordance with a portion of the sensor guide wire; and
identifying the one of the plurality of wires to which the current is applied based at least in part on the thermal image data.

30. The method of claim 26, further comprising:
exposing the core of the identified wire via a laser.

31. A kit, the kit comprising:
a first sensor guide wire according to claim 1;
a second sensor guide wire according to claim 1, wherein:
the plurality of electrodes of the first sensor guide wire are separated from one another by a first separation distance; and
the plurality of electrodes of the second sensor guide wire are separated from one another by a second separation distance that is different from the first separation distance; and
at least one computing device configured to receive respective signals from the plurality of wires of the first sensor guide wire or the second sensor guide wire.
